(19) United States
(12) Patent Application Publication
(10) Pub. No.: US 2008/0118930 A1
(43) Pub. Date: May 22, 2008

(54) Screening Method for Agents Capable of Reversing Fusidic Acid Resistance in Bacteria

(75) Inventors: Ian Chopra, West Yorkshire (GB); O'Neil Alexander, West Yorkshire (GB)

Correspondence Address: MYERS BIGEL SIBLEY & SAJOVEC, PO BOX 37428, RALEIGH, NC, 27627, US

(73) Assignee: LEO PHARMA A/S, Ballerup (DK)

(21) Appl. No.: 11/576,272

(22) PCT Filed: Oct. 14, 2005
(86) PCT No.: PCT/GB05/03937

§ 371 Date: Mar. 29, 2007

(30) Foreign Application Priority Data
Oct. 19, 2004 (GB) 0423140.3

(51) Int. Cl. G01N 33/53

(52) U.S. Cl. 435/7.1

(57) ABSTRACT

A screening method for agents which are capable of reversing resistance to the antibiotic fusidic acid in bacteria that contain the fusB resistance gene or a homologue thereof and that express the FusB fusidic acid resistance protein or a homologue thereof based on the interaction between the fusB resistance protein or a homologue thereof with elongation factor-G (EF-G) protein.

The present invention relates to screening methods for agents which are capable of reversing resistance to the antibiotic fusidic acid in bacteria, especially bacteria that contain the fusB resistance gene or a homologue thereof and that express the FusB fusidic acid resistance protein or a homologue thereof.

### BACKGROUND TO THE INVENTION

Fusidic acid is both a systemic and topical antibiotic that has proved effective in the treatment of severe diseases resulting from, in particular, staphylococcal infection. It is frequently prescribed in man for conditions such as osteomyelitis, endocarditis, skin and soft tissue infections, catheter-related infections, urinary tract infections, central nervous system shunt infections, surgical site infections, bacterial conjunctivitis and other bacterial infections of the eye, such as endophthalmitis. However, fusidic acid is most commonly employed in the outpatient setting as a topical antibiotic for treatment of superficial staphylococcal skin infections which include atopic eczema, impetigo and infected leg ulcers. It is also prescribed for veterinary use as an antibacterial agent.

Fusidic acid acts by selectively inhibiting prokaryotic protein synthesis through interference with dissociation of the translocase, elongation factor-G (EF-G), from the ribosome. Under normal conditions, the EF-G.GDP.ribosome ternary complex dissociates prior to entry of the next aminoacyl-tRNA complex into the ribosomal A-site. However, in the presence of fusidic acid, the ternary complex becomes stabilised, leading to inhibition of protein synthesis.

Fusidic acid has been successfully employed for over 40 years as an antibacterial agent, but its efficacy is now being compromised by the emergence of bacterial resistance.

Limited data on fusidic acid resistance in clinical isolates of S. aureus show that the resistance can arise from point mutations in the gene encoding EF-G (fusA) (Besier et al 2003, O'Neill et al, 2004 and Nagaev et al 2001), or by acquisition of an exogenous resistance determinant (fusB) that encodes a poorly characterised resistance mechanism (O'Brien et al 2002 and Projan 2000). The latter mechanism appears to represent the most important route to fusidic acid resistance in the clinical setting; a recent analysis of European fusidic acid-resistant clinical isolates of S. aureus has shown that fusB is present in an epidemic impetigo-causing strain which is widely spread in Europe (O'Neill et al 2004), and this determinant has also been. detected in S. aureus strains causing serious community-acquired infections (Witte et al, Euro surveillance, 1: 1-2, 2004).

Early studies into fusB-type resistance to fusidic acid were unable to identify modification of the protein synthetic apparatus or breakdown of fusidic acid (Chopra 1976, Sinden and Chopra, 1981), suggesting that resistance may be achieved by reduced ingress of fusidic acid into the cell. This led the author to suggest that exclusion of the antibiotic from the bacterial cell may result from the formation of a fusB-encoded permeability barrier.

In the present invention we have made significant advances towards elucidating the mechanism of action of the FusB protein, particularly through identification of a protein:protein interaction between FusB and EF-G which we believe is crucial to fusB-type resistance to fusidic acid. We have used this protein:protein interaction to develop an assay to screen for agents that could reverse resistance to the antibiotic fusidic acid in bacteria and also permit the design of targeted therapeutics directed to preventing this protein:protein interaction. The present invention therefore provides significant advances in combating fusidic acid resistant strains of bacterial and so will be of immense importance in the clinical environment.

### STATEMENT OF THE INVENTION

According to a first aspect of the invention there is provided an in vitro screening method for the identification of agents which modulate the interaction of FusB resistance protein or a homologue thereof with elongation factor-G (EF-G) in vitro comprising the steps of:
(i) providing a source of cells which express the FusB resistance protein or a homologue thereof;
(ii) adding at least one candidate agent to be tested and determining the effect or not, of said agent on the binding of the EF-G protein to the FusB protein or homologue thereof.

Throughout this specification and the claims which follow, unless the context requires otherwise, the word “comprise”, or variations such as “comprises” or “comprising”, will be understood to imply the inclusion of a stated integer or group of integers but not the exclusion of any other integer or group of integers.

Reference herein to a “FusB protein homologue” is intended to include a protein which has a similar function to the FusB protein identified in Staphylococcus aureus but which could be derived from another organism and which has a specified degree of sequence identity with the FusB protein (O'Brien et al 2002). A specified degree of homology could be 30% or more homology, preferably 40% or 60% or more homology, and more preferably 80% or more homology. The “sequence identity” of two amino acid sequences or of two nucleic acids is determined using the algorithm of Karlin and Altschul (Proc. Natl. Acad. Sci. USA 87:2264-2268, 1990), modified as in Karlin and Altschul (Proc. Natl. Acad. Sci. USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, scor=100, wordlength=12. BLAST protein searches are performed with the XBLAST program, score=50, wordlength=3. Where gaps exist between two sequences, Gapped BLAST is utilized as described in Altschul et al. (Nucleic Acids Res. 25:3389-3402, 1997). When utilizing BLAST and Gapped BLAST programs, the default parameters of the respective programs (e.g., XBLAST and NBLAST) are used. See http://www.ncbi.nlm.nih.gov.

Preferably, the FusB protein or homologue thereof and/or the EF-G protein is/are purified. The FusB protein or homologue thereof and/or the EF-G protein may be purified by any means known in the art, and conveniently by polyhistidine (his) tagging.

Preferably, one of the proteins, either the FusB protein or homologue thereof or the EF-G protein is immobilised.

Preferably, the FusB protein or homologue thereof or the EF-G protein is immobilised by for example and without limitation:
affinity tagging as described in Chapter 4 Einarson, M B and Orlinick, J R in “Protein—Protein Interactions”, edited by Erica Golemis published by Cold Spring Harbor Laboratory Press, Cold Spring Harbor, N.Y., 2002.
chemical cross-linking as described in Chapter 6 Nadeau, O W., Carlson, G M. in “Protein—Protein Interactions”, edited by Erica Golemis published by Cold Spring Harbor Laboratory Press, Cold Spring Harbor, N.Y., 2002.
antibody-based binding as described in Co-immunoprecipitation, Chapter 5, Adams, P., Seeholzer, S., and Ohh, M in “Protein—Protein Interactions”, edited by Erica Golemis published by Cold Spring Harbor Laboratory Press, Cold Spring Harbor, N.Y., 2002.

Any of the above mentioned techniques may be employed so that the protein:protein interaction may be observed in the presence of an agent capable of disrupting or reversing this interaction. It will be appreciated that it is the FusB resistance protein and EF-G protein interaction which is essential for the method of the present invention and thus the result of whether an agent is capable of disrupting the interaction is the same whether it is the FusB resistance protein or EF-G protein which is immobilised.

In one embodiment of tile invention, the FusB protein is a recombinant his-tagged FusB protein bound to an affinity matrix, and the step of determining the effect or not of an agent on the purified FusB resistance protein is achieved with immobilized FusB protein with EFG protein in whole cell lysates. Purified FusB resistance protein is bound to a metal affinity resin and washed with a staphylococcal cell lysate (which is a convenient source of EF-G), so as to bind EF-G. Putative therapeutic agents may be added either before or during this process. After repeated washing to remove EF-G that is unbound due to the presence of a putative therapeutic agent, the amount of EF-G remaining bound is determined in a qualitative fashion following SDS polyacrylamide gel electrophoresis (SDS PAGE).

In an alternative embodiment, the step of determining the effect or not of an agent on the purified immobilized FusB resistance protein with EFG protein is achieved by over-expressing and purifying recombinant affinity-tagged, ideally GST-tagged EF-G for addition to the assay in place of native EF-G from cell lysates. This embodiment would also allow use of commercially available enzyme-linked anti-affinity tag antibodies to enable detection of EF-G binding by an ELISA assay.

It will be appreciated that it would be also be possible to assess such protein:protein interactions without the need for immobilisation by techniques based on different parameters, for example the different sizes of the proteins. The FusB resistance protein has a molecular weight of 25 kDa, as compared to EF-G which is 80 kDa.

Thus, on the basis of the different sizes, EF-G could be retained on a membrane with a specified molecular weight cut-off above 25 kDa, for example and without limitation 50 kDa, thereby allowing FusB to traverse the membrane whilst EF-G is retained.

It will be appreciated by those skilled in the art that other methods for detecting protein:protein interactions of the FusB resistance protein with EF-G protein may also be used in the method of the present invention. Examples of such methods include, without limitation, include physical methods that detect a protein that binds to another such as affinity chromatography, affinity blotting, immunoprecipitation and methods based on cross-linking as hereinbefore described. In addition, library based methods can be employed such as protein probing as described in Chapter 9 Strich R in “Protein—Protein Interactions”, edited by Erica Golemis published by Cold Spring Harbor Laboratory Press, Cold Spring Harbor, N.Y., 2002, or phage display as described in Chapter 8, Goodyear C. S. and Silverman G. J. in “Protein—Protein Interactions”, edited by Erica Golemis published by Cold Spring Harbor Laboratory Press, Cold Spring Harbor, N.Y., 2002, or the two-hybrid system as described in Chapter 7, Serebriliskii I and Joung J. K. in “Protein—Protein Interactions”, edited by Eric& Golemis published by Cold Spring Harbor Laboratory Press, Cold Spring Harbor, N.Y., 2002.

It will be further appreciated that any method known in the art to detect the protein:protein interaction may be applied to the present invention so long as the interaction of the FusB resistance protein or homologues thereof and EF-G protein and agents that inhibit their interaction are detectable.

Preferably, the source of the FusB protein or homologue is a bacterial species, Gram positive or negative, in which the fusB resistance gene or a homologue is, or could be, present and which express the FusB resistance protein or homologue thereof and which may, as a result be resistant to the action of fusidic acid. The fusB or a homologue has to date been identified in S. aureus and other members of the genus Staphylococcus, and in Enterococcus spp, Listeria spp, Bacillus spp and Lactococcus spp. Accordingly any of these bacteria could provide a suitable source of FusB protein.

Preferably, in the instance where the bacteria is a Staphylococcus it is a species of Staphylococcus is selected from the group comprising S. caprae, S. gallinarum, S. aureus subsp. anaerobius, S. aureus subsp. aureus, S. epidermidis, S. haemolyticus, S. intermedius, S. lugdunensis, S. saccharolyticus,schleiferi subsp. schleiferi, S. xylosus, S. capitis subsp capitis, S. arlettae, S. warneri, S. hominis, S. stimulans, S. saprophtyticus, S. equorum, S. cohnii subsp. cohnii, S. auricularis, S. carnosus subsp. carnosus, S. kloosii, S. chromogenes, S. hyicus subsp. hyicus, S. pulvereri, S. felis, S. lentus, S. muscae and S. scuir, or any other Staphylococcus implicated in a mammalian bacterial infection.

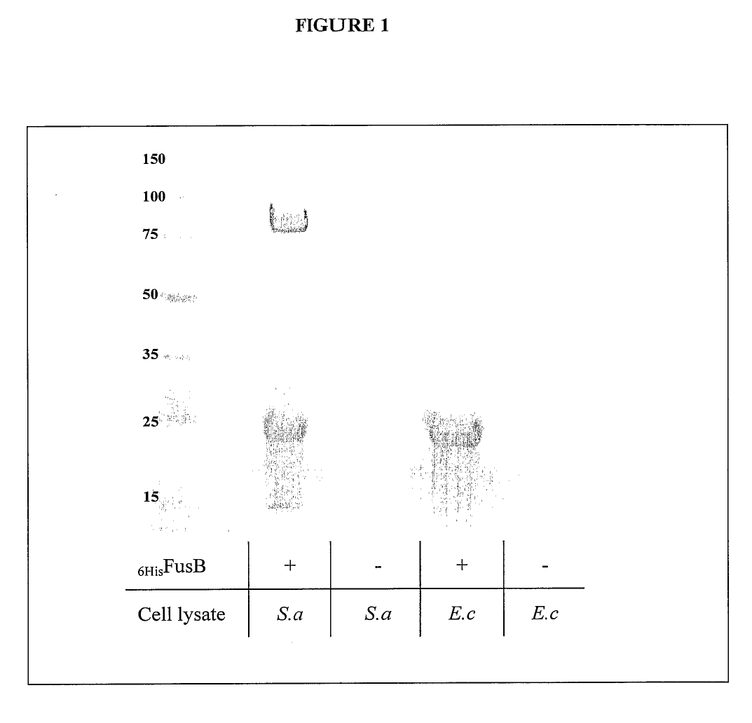

Cloning of fusB, and over-expression and purification of recombinant FusB has permitted an understanding, at the molecular level, of how this protein confers resistance to fusidic acid. Using cell-free coupled transcription-translation assays we have demonstrated that purified FusB, which is a soluble protein, protects the S. aureus translational apparatus from inhibition by fusidic acid in vitro. In contrast, the FusB protein appears unable to protect an E. coli cell-free system, consistent with our observations that expression of the fusB gene in E. coli fails to confer resistance to fusidic acid. Using immobilised FusB protein as an affinity matrix to identify cellular proteins with which tills resistance protein interacts, we have recently demonstrated that it binds to EF-G from S. aureus, but not E. coli (FIG. 1).

Bioinformatic analysis of FusB reveals no significant sequence homology with other proteins of proven function and consequently it is open to speculation how FusB protects the S. aureus translational apparatus. However, it appears that interaction with EF-G is central to the protective mechanism.

According to a second aspect of the invention there is provided a method for the screening and identification of an agent that prevents interaction of FusB protein or a homologue thereof with EF-G protein, and so blocks phenotypic expression of fusB mediated resistance in a bacterial cell.

Preferably, the method of the second aspect of the invention includes any one or more of the features of the method of the first aspect of the invention.

The invention will now be described by way of example only with reference to the following Figures wherein:

FIG. 1 shows the interaction between purified FusB (lower band) and native EF-G (upper band) from S. aureus lysates. No interaction occurs between FusB and the EF-G protein from E.coli.

### MATERIAL AND METHODS

### Cloning of fusB

PCR amplification of fusB was accomplished using primers 5′-GGTTGAAA CATATGAAAACAATGATTTAT (SEQ ID NO:1) and 5′-GTGGATCCCTTAATC TAGTTTATCA (SEQ ID NO:2). The PCR amplicon was ligated to pET28 (Novagen) via the engineered NdeI and BamHI restriction sites and and transformed into E. coli JM109. Transformants were selected on agar containing 50 μg kanamycin/ml and then screened for transformants containing inserts by restriction digest of plasmid preparations. Inserts were subjected to DNA sequencing to verify faithful PCR amplification and then transformed into E. coli BL21 (λDE3).

### Expression and Purification of Polyhistidine-Tagged FusB

BL21(λDE3) carrying pET28:fusB was grown in 2YT+0.5% glucose at 37° C. for 15 h with 50 μg kanamycin/ml. Cells were diluted 1 in 25 in 250 ml fresh 2YT with 30 μg kanamycin/ml and grown at 30° C. (220 rpm) until an OD₆₀₀ of 0.6 was reached. IPTG was added to 300 μM and incubation continued for 5 h. Cells were harvested at 5000×g for 15 min, pellets washed in phosphate buffered saline and frozen at −80° C. When required, cell pellets were thawed on ice for 20 min and then fully resuspended in 12 ml cold lysis buffer (50 mM sodium phosphate [pH 7], 300 mM NaCl, 1× BugBuster (Novagen; from 10× concentrate), 0.5 mM THP (Novagen), 5 mM imidazole, ¼ tablet of complete EDTA-free protease inhibitor cocktail (Roche)) on ice. Subsequently 90 KU rlysozyme and 1 KU benzonase (both from Novagen) were added and the mixture incubate in ice on a gently shaking platform for 20-30 min before centrifuging at 10,000×g for 20 minutes at 4° C. and collecting the supernatant in a fresh chilled tube. An aliquot (1 ml) of fully-equilibrated Talon metal affinity resin (Clontech) was added to the clarified sample and gently agitated in ice for 60 min to allow the polyhistidine-tagged protein to bind the resin. It was subsequently centrifuged at 700×g for 5 min at 4° C. and the supernatant removed. Resin was washed by adding 40 ml wash buffer (50 mM sodium phosphate [pH 7], 300 mM NaCl, 20 mM imidazole, 0.5 mM THP) and agitated gently on ice for 10 min. This was repeated and then 2 ml wash buffer was added to the resin, and resuspend by gentle vortexing. The resin was transferred to a 2-ml gravity-flow column, allowed to settle out of suspension and the buffer to drain. The column was washed once with 10 ml wash buffer and the polyhistidine-tagged protein eluted by adding 5 ml elution buffer (50 mM sodium phopshate [pH 7], 300 mM NaCl, 0.5 mM THP, 150 mM imidazole) to the column. The first 500 μl was discarded and the subsequent 1.5 ml eluate collected. This was dialysed at 4° C. using a 3.5K MWCO dialysis casette against 500 ml of 20 mM Tris HCl [pH 7.8], 300 mM NaCl, 1 mM DTT. After 3 h, the buffer was replaced with fresh buffer containing 50% glycerol and left overnight at 4° C. The presence and purity of polyhistidine-tagged protein was checked by analyzing 1-10 μl aliquots by electrophoresis through a 12% SDS polyacrylamide gel and quantified by Bradford assay.

### Poly-His Pull-Down

The protocol for the assay is conveniently referred to as a “pull-down assay” because it “pulls-down” interacting “prey” proteins using a “bait” protein.

### I Preparing Bait Protein

### 1) Equilibrate Immobilized Cobalt Chelate

1. Label enough Mini-Spin Columns (Pierce) to include sample, non-treated gel control and immobilized bait control.
2. For each Mini-Spin Column prepare 8 ml of wash buffer (25 mM Tris.HCl [pH 7.2] 150 mM NaCl, 1 mM MgCl₂, 0.5 mM THP, 100 μl protease inhibitor cocktail/1.25 ml) and add 80 μl 4 M imidazole.
3. Resuspend cobalt chelate by vortexing and place 50 μl of the slurry into each labeled Mini-Spin Column.
4. Add 400 μl of the wash solution to each spin column. Cap both column ends and invert several times to equilibrate
5. Remove both caps and place spin column in a Collection Tube.
6. Centrifuge at 1,250×g for 30 secs. Replace bottom cap. Discard wash solution.
7. Repeat wash steps X5

### 2) Immobilize Bait from Previously Purified Protein

1. Add 100-150 μg of polyhistidine-tagged bait protein (FusB) made up to 500 μl total volume in wash buffer
2. Replace bottom and top cap of each Mini-Spin Column
3. Incubate at 4° C. for at least 30 min with gentle rocking motion on a rotating platform.
4. Remove both caps from each spin column and place column in a Collection Tube.
5. Centrifuge at 1,250×g for 30 secs.
6. Replace bottom cap to spin column.
7. Add 400 μl of wash solution and wash X5 as before.

### II Prey Protein

### 1) Prepare and Capture Prey Protein (Steps 1 and 2 showed be done before the Day of the expt.)

1. Add 5 ml saturated RN4220 culture in TSB to 100 ml fresh broth and grow for 3 h at 37° C. with aeration
2. Harvest by centrifugation at 4° C. and wash in 5 ml cold Buffer A (25 mM Tris.HCl [pH 7.2] 150 mM NaCl)
3. Store pellet at −80° C.
4. Resuspend at 0.5 g/ml in cold buffer A
5. Add lysostaphin to 20 μg/ml and incubate with gentle mixing at 37° C. for 30 min
6. Centrifuge at 30,000×g for 30 min at 4° C.
7. Decant supernatant and add imidazole to 40 mM
8. Add up to 800 μl to immobilized fusion protein and control tubes (+resin/−bait protein)
9. Incubate at 4° C. for 1 hour with gentle agitation
10. Wash X5 as before.

### III. Bait-Prey Elution and Analysis

### 1. Boil Resin for 3 mins at 99° C. in 2 μl SDS Sample Buffer Supplemented with 50 mM EDTA and Run Directly on SDS-PAGE Gel (10-12%)

### EXAMPLE 1

With reference to FIG. 1 there is shown the FusB and EF-G interaction. The upper band that can be seen in the second lane is EF-G, as confirmed by mass spectrometry on tryptic digests of this protein band. Using cell-free coupled transcription-translation assays we have demonstrated that purified FusB, which is a soluble protein, protects the S. aureus translational apparatus from inhibition by fusidic acid. In contrast, the FusB protein appears unable to protect an E. coli cell-free system consistent with earlier observations that expression of the fusB gene in E. coli fails to confer resistance to fusidic acid. Using immobilised FusB protein as an affinity matrix we have recently demonstrated that it binds to EF-G from S. aureus, but not E. coli. (refer to lanes 4 and 5 of FIG. 1). Lanes 3 and 5 are control lanes that show that no non-specific binding of EF-G to the metal affinity resin occurs in the absence of FusB.

### REFERENCES

Besier S, Ludwig A, Brade V and Wichelhaus (2003) Molecular analysis of fusidic acid resistance in Staphylococcus aureus. Mol Microbiol 47, 463-9
Chopra I (1976) Mechanisms of resistance to fusidic acid in Staphylococcus aureus. J Gen Microbiol 96, 229-38.
Nagaev I, Bjorkman D, Andersson I and Hughes D. (2001) Biological cost and compensatory evolution in fusidic acid-resistant Staphylococcus aureus. Mol Microbiol 40, 433-39.
O'Brien F, Price C, Gribb W and Gustafson J (2002) Genetic characterisation of fusdic acid and cadmium resistance determinants of Staphylococcus aureus plasmid pUB101. J Antimicrob Chemother 50, 313-21.
O'Neill A J, Larsen A R, Henriksen A S and Chopra I. (2004) An epidemic fusidic acid-resistant strain of Staphylococcus aureus carries the fusB determinant, whereas mutations in fusA are prevalent in other resistant isolates. Antimicrobial Agents and Chemotherapy, 48.
Projan S (2000) Antibiotic Resistance in the Staphylococci. In Fischetti V, Novick R, Ferretti J, Portnoy D and Rood J (eds), Gram-positive pathogens. American Society for Microbiology, Washington D.C.
Sinden, D. & Chopra, I. (1981). Fusidic acid resistance in Staphylococcus aureus. In: Staphylococci & Staphylococcal Infections. (Jeljaszewicz, J., Ed), pp. 571-574. Gustav Fischer Verlag, Stuttgart-New York.
Witte et al, Euro surveillance, 1:1-2,2004

1. An in vitro screening method for the identification of agents which modulate the interaction of FusB resistance protein or a homologue thereof with elongation factor-G (EF-G) in vitro comprising:
(i) providing a source of cells which express the FusB resistance protein or a homologue thereof; and
(ii) adding at least one candidate agent to be tested and determining the effect or lack thereof, of said agent on the binding of the EF-G protein to the FusB protein or homologue thereof.

2. The method according to claim 1, wherein the FusB protein or homologue thereof and/or the EF-G protein is/are purified.

3. The method according to claim 1, wherein at least one of the FusB protein or homologue thereof or the EF-G protein is immobilised.

4. The method according to claim 3, wherein the FusB protein or homologue thereof or the EF-G protein is immobilised by a technique selected from the group consisting of affinity tagging, chemical cross-linking and antibody-based binding so that the protein:protein interaction is observable in the presence of an agent capable of disrupting or reversing this interaction.

5. The method according to claim 1, wherein the FusB protein is a recombinant polyhistidine-tagged FusB protein.

6. The method according to claim 5, wherein the polyhistidine-tagged FusB protein is bound to an affinity matrix, and the step of determining the effect or lack thereof of an agent on the purified FusB resistance protein is with immobilized FusB protein.

7. The method according to claim 1, wherein the EFG protein is derived from whole cell lysates.

8. The method according to claim 7, wherein unbound EF-G is removed by washing and the amount of EF-G remaining bound due to the presence of a candidate/putative therapeutic agent is determined.

9. The method according to claim 8, wherein the EF-G remaining bound due to the presence of a candidate/putative therapeutic agent is determined qualitatively following SDS polyacrylamide gel electrophoresis (SDS PAGE).

10. The method according to claim 1, wherein the source of EFG protein is from over-expressing and purifying recombinant affinity-tagged EFG.

11. The method according to claim 10, wherein the EFG is GST-tagged.

12. The method according to either claim 10, further including the step of using enzyme-linked anti-affinity tag antibodies to enable detection of EF-G binding by an ELISA assay.

13. The method according to claim 1, wherein EF-G is retained on a membrane with a specified molecular weight above about 25 kDa thereby allowing FusB to traverse the membrane whilst EF-G is retained so that the interaction of FusB resistance protein or a homologue thereof with EF-G in the presence of a candidate/putative therapeutic agent can be determined.

14. The method according to claim 1, wherein the technique for detecting protein:protein interactions of the FusB resistance protein with EF-G protein in the presence of a candidate/putative therapeutic agent is selected from the group consisting of affinity chromatography, affinity blotting, immunoprecipitation, chemical-cross-linking, protein probing, phage display and the two-hybrid system.

15. The method according to any preceding claim 1, wherein the source of the FusB protein or homologue is a Gram positive or negative bacterial species, in which the fusB resistance gene or a homologue is capable of expressing the FusB resistance protein or homologue thereof.

16. The method according to claim 15, wherein the-bacterial species is selected from the group consisting of the genus Staphylococcus, Enterococcus spp, Listeria spp, Bacillus spp and Lactococcus spp.

17. The method according to claim 15 wherein the species or subspecies of Staphylococcus is selected from the group consisting of S. caprae, S. gallinarum, S. aureus subsp. anaerobius, S. aureus subsp. aureus, S. epidermidis, S. haemolyticus, S. intermedius, S. lugdunensis, S. saccharolyticus,schleiferi subsp. schleiferi, S. xylosus, S. capitis subsp capitis, S. arlettae, S. warneri, S. hominis, S. stimulans, S. saprophyticus, S. equorum, S. cohnii subsp. cohnii, S. auricularis, S. carnosus subsp. carnosus, S. kloosii, S. chromogenes, S. hyicus subsp. hyicus, S. pulvereri, S. felis, Slentus, S. muscae and S. scuir.

18. A method for the screening and identification of an agent that prevents interaction of FusB protein or a homologue thereof with EF-G protein, and so blocks phenotypic expression of fusB mediated resistance in a bacterial cell.

19. (canceled)


SEQUENCE LISTING (2 sequences)

SEQ ID NO: 1 - DNA, 28 bp, Artificial
  PCR primer
gttgaaacatatgaaaacaatgatttat

SEQ ID NO: 2 - DNA, 25 bp, Artificial
  PCR primer
gtggatcccttaatctagtttatca